(19) United States
(12) Patent Application Publication
(10) Pub. No.: US 2006/0271065 A1
(43) Pub. Date: Nov. 30, 2006

(54) CATHETER CAPTURE DEVICE

(76) Inventor: Kenneth A. High, Helena, MT (US)

Correspondence Address: ANTOINETTE M. TEASE, P. O. BOX 51016, BILLINGS, MT, 59105, US

(21) Appl. No.: 11/426,315

(22) Filed: Jun. 25, 2006

Related U.S. Application Data
(62) Division of application No. 10/837,879, filed on May 3, 2004.
(60) Provisional application No. 60/466,959, filed on May 1, 2003.

(51) Int. Cl. A61F 11/00

(52) U.S. Cl. 606/108

(57) ABSTRACT

A Foley catheter capture device including a tube with an interiorly extending flange. The catheter is captured by inflating the balloon portion of the Foley catheter inside the tube beyond the flange. A second Foley catheter capture device includes a pair of clamshell halves which are fitted together to form a tubular structure. One of the clamshell halves has a pin secured thereto extending inwardly where the pin is sized to be received by the laterally extending hole of the Foley catheter. A Councill catheter capture device is described which includes a wire having a nodule secured to the wire intermediate the ends thereof. The nodule is sized to be larger than the axial hole of the Councill catheter whereby the catheter is captured when an end of the wire is threaded through the axial hole with the nodule positioned inside the catheter.

### CROSS-REFERENCE TO RELATED APPLICATION

This application is a divisional of U.S. patent application Ser. No. 10/837,879, filed on May 3, 2004. That application in turn claims the benefit of U.S. patent application Ser. No. 60/466,959, filed on May 1, 2003.

### BACKGROUND OF THE INVENTION

The present invention relates to a catheter capture device for use with an urethral sound when positioning a suprapubic catheter within the bladder of a patient.

Suprapubic catheter insertion techniques are well known where a urethral sound is advanced through the urethra, through the bladder wall and then through the abdominal wall and skin. Several methods are known for attaching a catheter to the exposed urethral sound and then using the urethral sound to position the catheter at a location inside the bladder. Three of such methods are shown in U.S. Pat. No 5,843,113 to Kenneth High, U.S. Pat. No. 5,152,749 to Giesy et al., and U.S. Pat. No. 5,348,541 to Lyell.

The present invention is an improvement of the known techniques for capturing a suprapubic catheter with a urethral sound prior to placement of the catheter inside the bladder.

### SUMMARY OF INVENTION

Several embodiments of a catheter capture device are described. In a first embodiment, a capture device is attached to an urethral sound to capture a Foley catheter. This device includes an elongate tube having a first end attached to the urethral sound and a second open end in which is located an interiorly extending annular flange. The flange is sized to prevent the catheter end form being removed from the elongate tube when the balloon end of the Foley catheter is inserted in the tube and the balloon portion inflated.

A second capture device is described which is attached to an urethral sound and includes a pair of clamshell halves which are fitted together to form an elongate tubular structure. This tubular structure has one end attached to the urethral sound and one of the clamshell halves has a pin secured thereto extending inwardly. The pin is used to extend through the laterally extending hole of the Foley catheter to capture the catheter.

A third capture device is described for capturing a Councill catheter. This embodiment includes a wire having a nodule secured to the wire intermediate the ends thereof and the nodule sized to have a dimension larger than the diameter of the axially aligned hole of the Councill catheter whereby the catheter is captured when an end of the wire is threaded through the hole provided in the catheter with a nodule positioned inside the catheter.

### DESCRIPTION OF THE DRAWINGS

In order that the invention may be clearly understood and readily carried into effect, and preferred embodiment of the invention will now be described, by way of example only, with reference to the accompanying drawings wherein:

FIG. 1 is a perspective view of a tip end of a conventional Foley catheter;

FIG. 2 is perspective view of a balloon capture collar according to the present invention;

FIG. 3 is a cross-sectional view of a balloon capture collar shown in FIG. 2;

FIG. 4 is the cross-sectional view shown in FIG. 3 with a Foley catheter inserted prior to a balloon being inflated;

FIG. 5 is the cross-sectional view shown in FIG. 4 with the balloon inflated;

FIG. 6 is a perspective view of a second embodiment showing a clamshell capture head;

FIG. 7 is a perspective view shown in FIG. 6 with portions cut away;

FIG. 8 is an exploded view of the invention shown in FIG. 6 with positioned cut away and showing a Foley catheter positioned within one of two clamshell halves;

FIG. 9 is a perspective view as shown in FIG. 8 with the two clamshell halves partially closed together;

FIG. 10 is a perspective view shown in FIG. 8 with the two clamshell halves closed together;

FIG. 11 is a perspective view of a conventional urethral sound with a removable tip removed therefrom;

FIG. 12 is a perspective view of a fourth embodiment of a catheter capture device according to the present invention;

FIG. 13 is a schematic view showing the catheter capture device shown in FIG. 12 with a catheter;

FIG. 14 is a schematic view showing the catheter capture device with a catheter as shown in FIG. 13 inserted in a urethral sound according to the present invention;

FIG. 15 is a perspective view of a fifth embodiment of an attach capture device according to the present invention;

FIG. 16 is a schematic view showing the catheter capture device shown in FIG. 15 with a catheter; and

FIG. 17 is a schematic view showing the catheter capture device with a catheter as shown in FIG. 16 inserted in a urethral sound according to the present invention.

### DESCRIPTION OF A PREFERRED EMBODIMENT

The present invention relates to several embodiments of a catheter capture device for use with an endocystotomy tool, such as is described in U.S. Pat. No. 5,843,113 (hereinafter the '113 patent). A urethral sound 60, as described in the '113 patent, is shown in FIG. 11. The urethral sound 60 includes a finger attachment 62 for manipulating the urethral sound 60 and includes a distal end 64 having internal female threads 66 provided at the extreme end thereof. The female threads 66 are sized to receive the male threads 68 of a removable tip 70. The urethral sound 60 is further provided with a hole 72 extending laterally through the urethral sound 60 in a direction substantially perpendicular to the longitudinal axis of urethral sound 60.

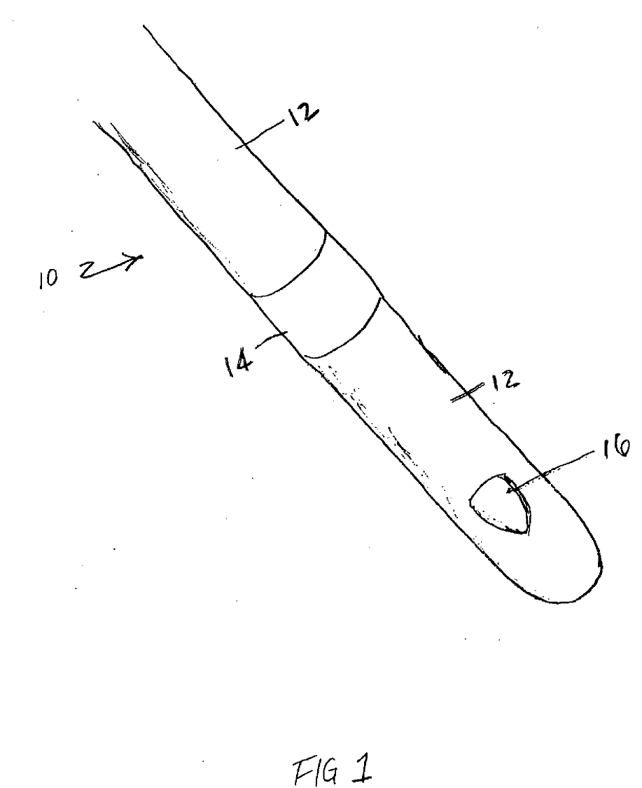

A conventional Foley catheter 10 is shown in FIG. 1. Only a tip of the Foley catheter is shown in FIG. 1. The tip includes a catheter tubular portion 12 which extends into a tubular inflatable balloon portion 14. A distal end of the balloon portion 14 extends into a continuation of the tubular catheter 12, as shown. A hole 16 is provided at the tip of the Foley catheter, and the hole 16 extends through the tubular wall of the tubular portion 12.

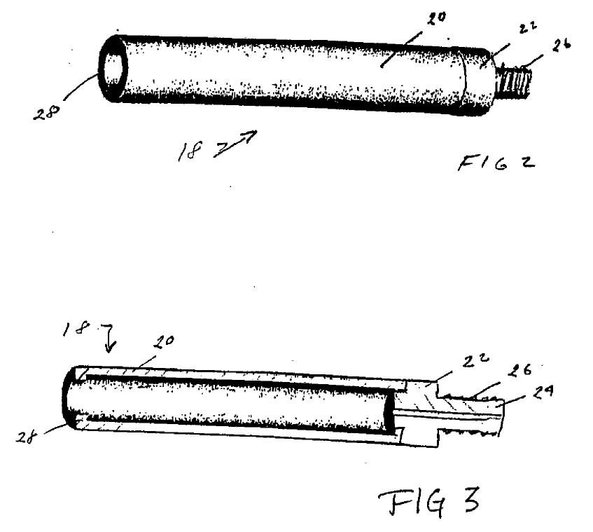

A first embodiment of the present invention including a balloon capture collar 18 is shown in FIGS. 2-5. The balloon capture collar 18 includes a tubular wall 20, as shown in FIG. 3. A plug 22 is secured to the wall 20 at an end thereof for closing an open end of tubular wall 20. The plug 22 has a cylindrical extension 24 having male threads 26. The male threads 26 correspond with the male threads 68, shown in FIG. 11, attached to a removable tip 70, and are used for threadably attaching the capture collar 18 to the urethral sound 60, with female threads 66 located at a distal end 64 of the urethral sound 60.

At an end of the balloon capture collar 18 opposite the plug 22, and interiorly extending annular flange 28 is provided, as shown in FIG. 3.

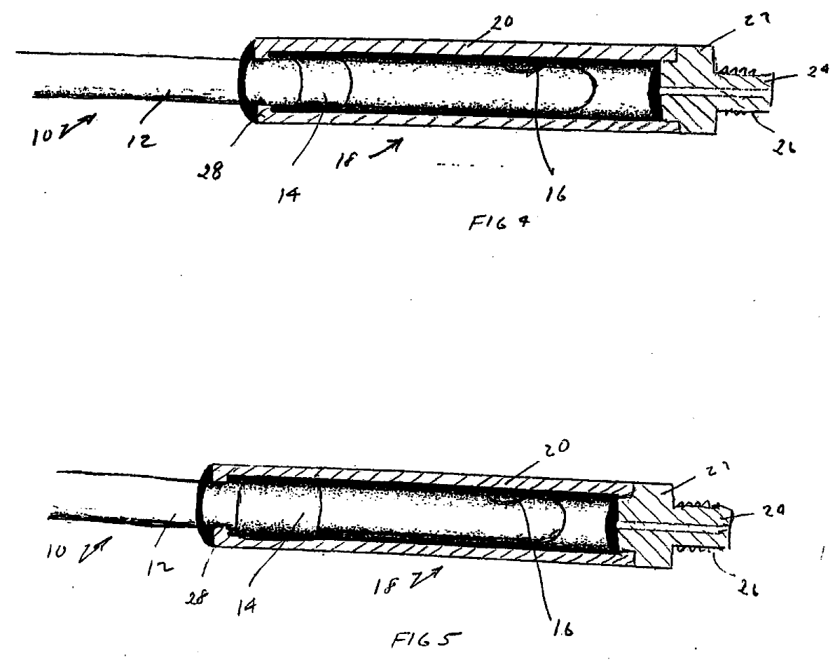

FIG. 4 shows the balloon capture collar 18 with the tip of a Foley catheter 10 having its tip positioned in the interior of the tubular wall 20, before the balloon portion 14 has been expanded or inflated. FIG. 5 shows the balloon capture collar 18 with the balloon portion 14 of the Foley catheter 10 inflated. The flange prevents the inflated balloon portion 14 from being retracted through the flange 28. Flange 28, however, is not necessary for operation of this device, and it is contemplated that balloon capture collar 18 could be used equally as well without the flange 28.

A clamshell capture tool 30, according to a second embodiment of the present invention, is shown in FIGS. 6-10. The clamshell capture tool includes opposed clamshell halves 32 and 34. The clamshell half 32 has an extension 36 having a semicircular shape and having exterior male threads 26. The other clamshell half 34 has an extension 38 which also has a semicircular shape identical with the shape extension 36. The extension 38 also has exterior male threads 26 which conform with the male threads of extension 36. The threads 26 allow the clamshell capture tool 30 to be threaded into the female threads 66 of the urethral sound 60.

The clamshell halves 32 and 34 are shaped so that when the halves are closed together, a generally tubular structure is provided, as shown in FIG. 6.

The clamshell half 34 is provided with a forward pin 40 and a rearward pin 42. The pins 40 and 42 are aligned and spaced apart in parallel relation and extended in a direction perpendicular to a longitudinal axis of clamshell half 34.

The clamshell half 32 is provided with a forward hole 44 for receiving an end of the pin 40, and is further provided with a rearward hole 46 for receiving the pin 42.

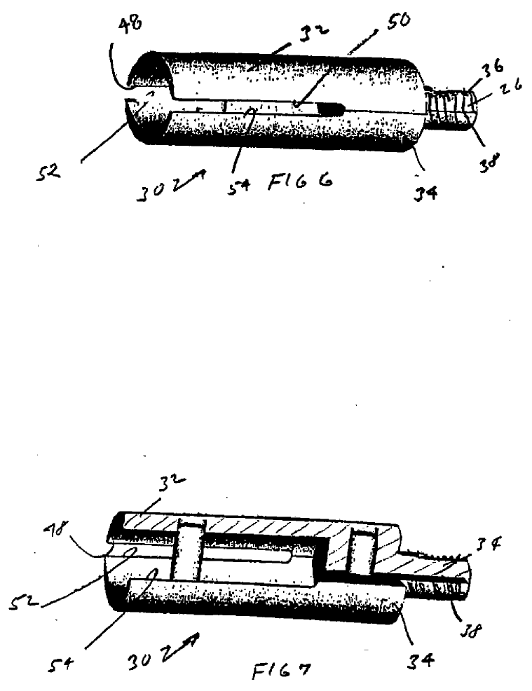

The clamshell half 32 is further provided with a pair of longitudinally-extending cut away portions 48 and 50, as shown in FIGS. 6 and 7. Similarly, the clamshell half 34 is provided with a pair of cutaway portions 52 and 54. The cutaway portions 48-54 are sized and positioned so that they form a pair of slots on either side of the clamshell capture tool 30, as shown in FIG. 6.

In operation, the clamshell half 32 is removed from the clamshell half 34, as shown in FIGS. 8 and 9. The catheter 10 is then positioned within the clamshell half 34 with the hole 16 of the Foley catheter 10 receiving the forward pin 40.

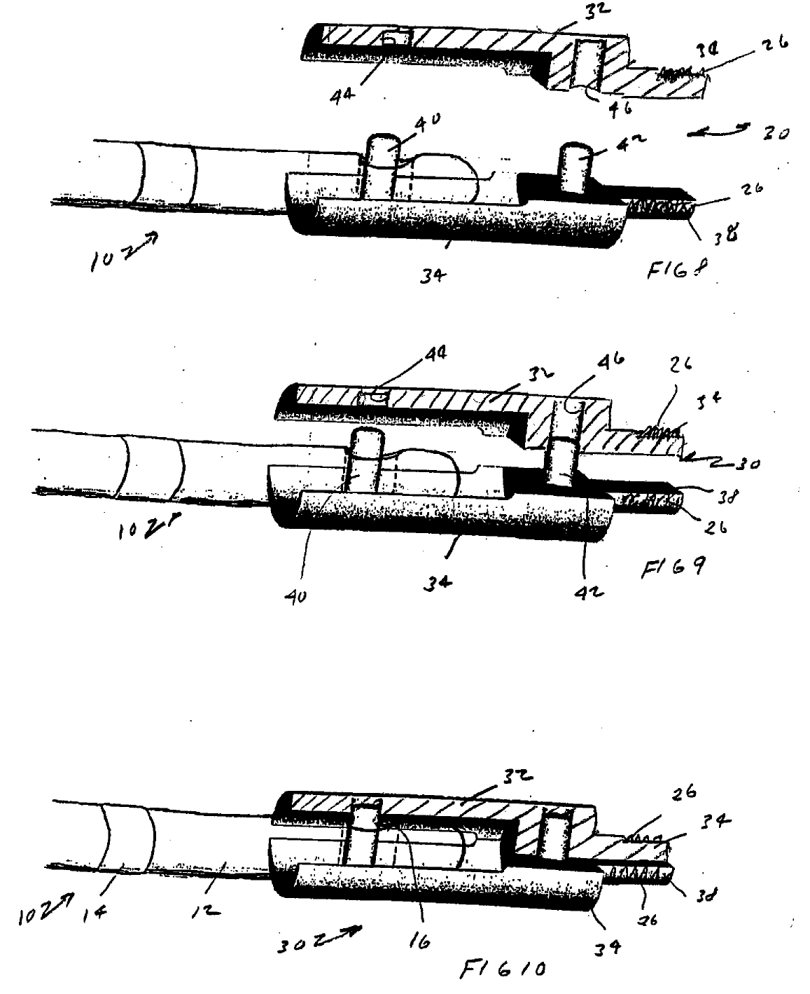

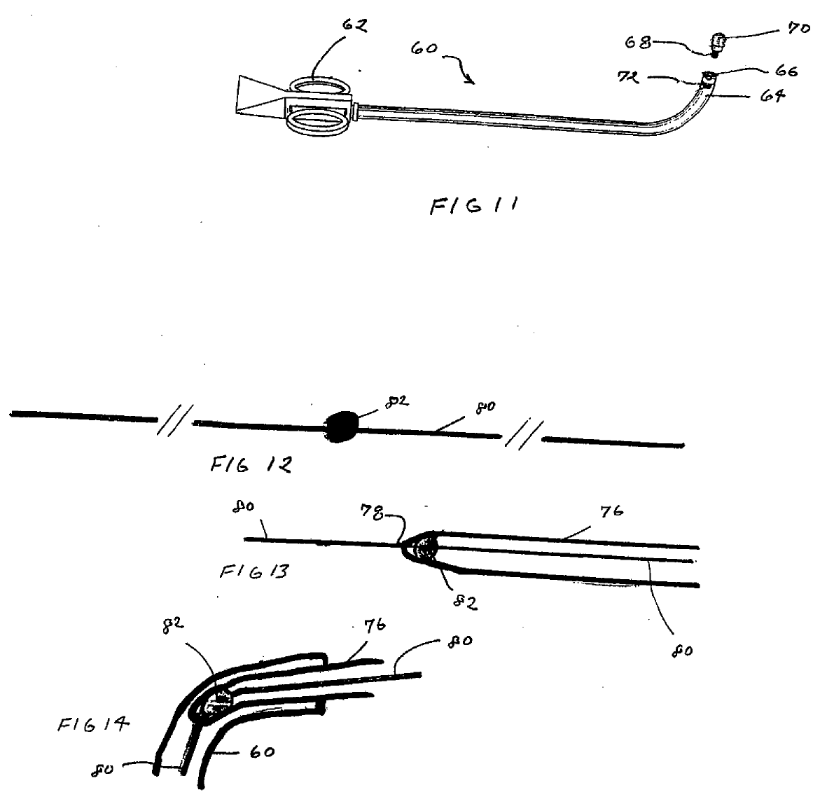

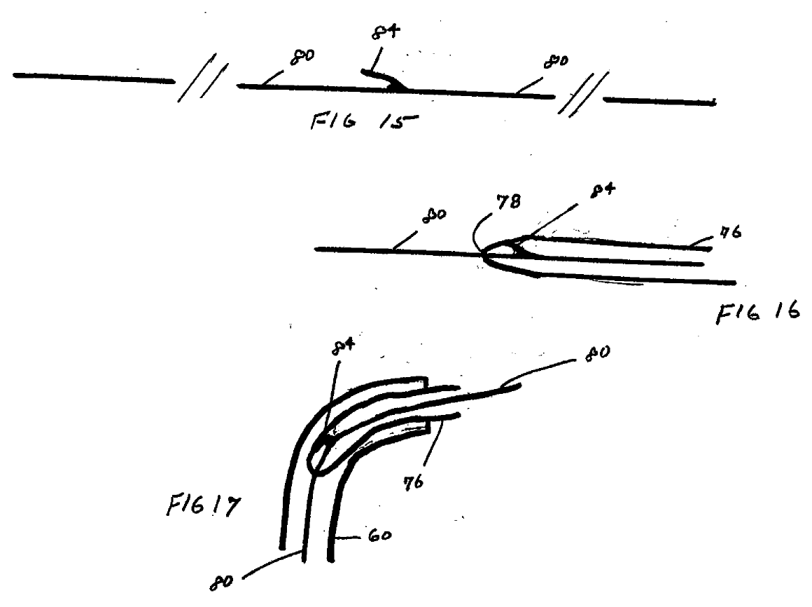

Next, the clamshell half 32 is fitted over the clamshell half 34 with the pins 40 and 42 being received by the holes 44 and 46, respectively, as shown in FIGS. 9 and 10. The pins 40 and 42 are sized to frictionally fit within holes 44 and 46, respectively, so that when the two clamshell halves are fitted together, they are held together by the frictional fit until they are to be intentionally removed.

In using the present invention, the urethral sound 60 is advanced through the abdominal wall and skin of a patient in a conventional manner, as described in the '113 patent. The removable tip 70 is unscrewed from the distal end 64 of the urethral sound 60, and a pin or other holding device is passed through the hole 72 to secure the urethral sound 60 outside the abdominal wall while the surgeon secures a catheter to the urethral sound 60.

The present invention is used to secure the Foley catheter to the end of urethral sound 60. In a first embodiment of the present invention, the balloon capture collar 16 is threaded into threads 66 of the urethral sound 60. Next, the Foley catheter is inserted into the balloon capture collar 18, as shown in FIG. 4, and the balloon 14 inflated to secure the tip end of the Foley catheter within the balloon capture collar 18.

With the second embodiment, the clamshell collar 30 is threaded into threads 66 of the urethral sound 60. Next, the Foley catheter tip is positioned over the clamshell half 34 with the hole 16 of the Foley catheter inserted over the pin 40, as shown in FIG. 9 of the present application. The second clamshell half 32 is then fitted over the clamshell half 34 with the pin 40 being received by hole 44 and the pin 42 being received by hole 46. In this manner, the Foley catheter tip is held within the clamshell capture tool.

As described in the '113 patent, the urethral sound 60 is then retracted through the bladder and urethral meatus with the attached catheter. With the balloon device, the balloon 14 is deflated and the catheter tip removed from the urethral sound 60. With the clamshell head, the clamshell half 32 is removed from the clamshell half 34, and the catheter tip removed from the pin 40.

After accomplishing this, the catheter is withdrawn back into the bladder, the balloon inflated, the catheter irrigated and the catheter secured.

Further, it is contemplated in a third embodiment of the present invention that the balloon portion 14 could be inflated within the distal end 64 of the urethral sound 60 after the tip 70 is removed. The balloon portion 14 would be inflated after inserting the Foley catheter 10 into the end 64 of urethral sound 60 approximately 8-10 cm. In this embodiment, it would be unnecessary to secure the balloon capture collar 18 or the clamshell capture tool 30 to the distal end 64 of the urethral sound 60 before inflating the balloon portion 14.

A fourth embodiment of the present invention is shown in FIGS. 6-8. This embodiment is used with a Foley catheter 66 having a hole 68 in the distal end of the catheter. This type of catheter is also known as a Concill catheter. This embodiment includes a wire 80 to which is attached a ball 82 intermediate the ends of the wire 80, as shown in FIG. 6. In using the present invention, the wire 80 is threaded through the catheter 66 with the ball 82 positioned inside the catheter 66. The wire is then pulled so that the ball 82 is positioned adjacent the hole 68. The ball 82 is sized to have a diameter larger that the diameter of the hole 68 so that the ball 82 cannot be pulled through the hole 68, as shown in FIG. 7. In using this fourth embodiment, the urethral sound 60 is again extended outside the abdominal wall of a patient. The wire 80 is then threaded through the urethral sound 60 and releasably structured thereto. Next, the urethral sound 60 is then retracted through the bladder and urethral meatus with the attached catheter until the catheter is within the bladder, the wire 80 is then released and the urethral sound 60 removed, leaving the catheter 66 within the bladder. The balloon is then inflated, the catheter irrigated, and the catheter secured.

A fifth embodiment of the present invention is shown in FIGS. 9-11. This fifth embodiment is also used with a Councill catheter 76, as with the fourth embodiment. This embodiment is shown in FIG. 9 and includes a wire, but instead of a ball 82, a rigid hook 84 is used to engage the inner wall of catheter 76, as was the case with the forth embodiment and as shown in FIG. 10. The hook with wire is inserted in the catheter, as shown in FIG. 10, and the catheter with the wire 80 is inserted in the urethral sound 60, as with the fourth embodiment. This fifth embodiment is used in the same manner as the fourth embodiment.

While the fundamental novel features of the invention have been shown and described, it should be understood that various substitutions, modifications, and variations may be made by those skilled in the art, without departing from the spirit or scope of the invention. Accordingly, all such modifications or variations are included in the scope if the invention as defined by the following claims.

I claim:

1. A device for capturing a catheter having a hole at an end thereof aligned with the longitudinal axis of the catheter comprising:
a wire; and
a nodule secured to the wire intermediate the ends thereof;
the nodule sized to have a dimension larger than the diameter of the hole;
whereby the catheter is captured when an end of the wire is threaded through the hole provided in the catheter with the nodule positioned inside the catheter.

2. The device according to claim 1 wherein the nodule comprises a ball.

3. The device according to claim 1 wherein the nodule comprises a hook.

4. A catheter capture device used in combination with a urethral sound and a catheter having a hole at an end thereof aligned with the longitudinal axis of the catheter, the catheter capture device comprising:
a wire having one end releasably secured to the urethral sound; and
a nodule secured to the wire intermediate the ends thereof;
the nodule sized to have a diameter larger then the diameter of the hole;
whereby the catheter is captured when the end of the wire releasably secured to the urethral sound is first threaded through the hole provided in the catheter with the nodule positioned inside the catheter before releasably securing the end of the wire to the urethral sound.
